(19) United States
(12) Patent Application Publication
(10) Pub. No.: US 2026/0078385 A1
(43) Pub. Date: Mar. 19, 2026

(54) PROBIOTICS DESIGNED TO EXPRESS AND SECRETE AKKERMANSIA MUCINIPHILA TARS, AND A VECTOR FOR PRODUCING THE SAME

(71) Applicant: Korea Researh Institute of Bioscience and Biotechnology, Daejeon (KR)

(72) Inventors: Myung Hee KIM, Daejeon (KR); Shinhye Park, Daejeon (KR); Su Man Kim, Daejeon (KR); Seungho Hwang, Daejeon (KR)

(73) Assignee: Korea Research Institute of Bioscience and Biotechnolgy, Daejeon (KR)

(21) Appl. No.: 19/401,917

(22) Filed: Nov. 26, 2025

Related U.S. Application Data
(63) Continuation of application No. PCT/KR2024/007468, filed on May 31, 2024.

(30) Foreign Application Priority Data
May 31, 2023 (KR) 10-2023-0069812

(51) Int. Cl. C12N 15/71; A61K 9/00; A61K 35/741; A61P 29/00; C12N 1/20; C12N 9/22; C12N 15/113; C12N 15/72; C12R 1/19

(52) U.S. Cl. CPC C12N 15/71; A61K 9/0053; A61K 35/741; A61P 29/00; C12N 1/205; C12N 9/226; C12N 15/113; C12N 15/72; C12N 2310/20; C12R 2001/19

(57) ABSTRACT

The present invention relates to: a transformed strain expressing and secreting Akkermansia muciniphila TARS (AmTARS); and a vector for producing same. More specifically, the present invention relates to an Escherichia coli Nissle 1917 (EcN) strain expressing AmTARS, a composition comprising same, a novel vector for producing same, and an EcN transformation method using same.

### CROSS-REFERENCE TO RELATED APPLICATIONS

This application is a By-Pass Continuation of International Application No. PCT/KR2024/007468 filed May 31, 2024, claiming priority based on Korean Patent Application No. 10-2023-0069812 filed May 31, 2023, the entire disclosures of which are incorporated herein by reference.

### INCORPORATION BY REFERENCE OF SEQUENCE LISTING

The instant application contains a Sequence Listing which has been filed electronically in xml format and is hereby incorporated by reference in its entirety. Said xml file, created on Nov. 26, 2025, is named Q315935_sequence listing as filed.xml and is 14,863 bytes in size.

### TECHNICAL FIELD

The present invention relates to a transformed strain expressing and secreting TARS (AmTARS) of Akkermansia muciniphila and a vector for producing the same, and more specifically, to an Escherichia coli Nissle 1917 (EcN) strain expressing AmTARS, a composition comprising the same, a novel vector for producing the same, and a method for transforming EcN using the same.

### BACKGROUND ART

The Escherichia coli Nissle 1917 (EcN) strain, well-known as a probiotics, is one of the most extensively studied strains worldwide and has been researched for the prevention and alleviation of various gastrointestinal diseases such as inflammatory bowel disease (IBD), constipation, and irritable bowel syndrome. EcN is a non-pathogenic strain lacking factors that exhibit cytotoxicity and is known to function by influencing intestinal epithelial cells to induce the secretion of beta-defensin, an antimicrobial peptide, thereby inhibiting pathogenic microorganisms. Currently, it is also manufactured as Mutaflor capsules (a freeze-dried product of EcN) and sold as a prescription drug in Korea.

IBD is a chronic inflammatory disease that includes ulcerative colitis and Crohn's disease. Although the exact cause is not known, it is believed to result from genetic and environmental factors. While various treatments can alleviate symptoms, no definitive cure has been developed to date. Studies comparing the gut microbiota of healthy individuals and IBD patients have shown that the proportion of strains commonly known as beneficial bacteria is significantly lower in IBD patients compared to healthy individuals. Based on this, various research teams have published papers on the results of studies using probiotics to alleviate IBD symptoms, and active research is still ongoing. Aminoacyl-tRNA synthetase (ARS) is an essential enzyme for protein synthesis that links amino acids to tRNA. Some ARS enzymes are known to perform various functions both inside and outside cells, in addition to their inherent role in protein synthesis. Among them, threonyl-tRNA synthetase (TARS) derived from Akkermansia muciniphila (AmTARS), a beneficial gut microbiome bacterium, has been found to be secreted into the gut environment, where it monitors inflammatory conditions in the gut and interacts with macrophages during inflammation to perform anti-inflammatory functions. Secreted AmTARS interacts with Toll-like receptor 2 (TLR2), an immune receptor of macrophages, to activate downstream MAPK and PI3K/AKT signaling pathways, thereby promoting the differentiation of M2 macrophages. Ultimately, it enhances the activity of the transcriptional regulatory protein CREB, inducing the secretion of the anti-inflammatory cytokine IL-10. Through this mechanism, it alleviates inflammation and plays a role in maintaining immune homeostasis in the gut.

Meanwhile, ARS (aminoacyl-tRNA synthetase) is an enzyme that links amino acids to transfer RNA (tRNA) during the initial stage of protein synthesis. Among human-derived AARS proteins, some are known to be secreted from cells and perform various functions. Specifically, human TARS (threonyl-tRNA synthetase) has been reported to induce angiogenesis mediated by inflammatory signals when secreted outside the cell and to increase the synthesis of MUC1 (Mucin 1), one of the major proteins of mucin containing a high amount of threonine, in pancreatic cancer cells. Additionally, it has been reported that this protein functions as a key component of the vertebrate-specific protein synthesis initiation complex, which is essential for the synthesis of proteins encoded by genes required for the development of blood vessels and the nervous system during the evolutionary transition from invertebrates to vertebrates.

On the other hand, it has been confirmed that Akkermansia muciniphila, a gut microorganism reported to play an important role in metabolic diseases through human immune regulation, specifically secretes TARS, and that this TARS promotes the secretion of IL-10 by macrophages, thereby activating B cells and inducing anti-inflammatory immune homeostasis. However, since Akkermansia muciniphila is difficult to culture and proliferate in vitro, there are limitations in utilizing the strain itself for the prevention or treatment of inflammatory diseases.

### Problem to be Solved

Accordingly, the present inventors have conducted repeated research to develop a transformed strain that not only stably produces and secretes AmTARS but is also safe for consumption. As a result, they confirmed that when EcN is transformed using a vector consisting of specific components and sequences, a strain capable of stably producing and secreting AmTARS can be manufactured, thereby completing the present invention. Therefore, an object of the present invention is to provide an Escherichia coli Nissle 1917 (EcN) strain transformed with a vector comprising a nucleic acid encoding TARS of Akkermansia muciniphila (AmTARS).

Another object of the present invention is to provide a composition for preventing, improving, or treating inflammatory diseases comprising the transformed EcN strain. Another object of the present invention is to provide a vector comprising a 5′-homology arm, a promoter, a ribosome binding site consisting of the nucleotide sequence of SEQ ID NO: 2 (AGGAGGT), a nucleic acid encoding TARS of Akkermansia muciniphila (AmTARS), and a 3′-homology arm,
wherein the 5′-homology arm comprises a nucleotide sequence corresponding to a portion of the exo gene and exo/cea intergenic region of Escherichia coli Nissle 1917 (EcN),
and the 3′-homology arm comprises a nucleotide sequence corresponding to a portion of the exo/cea intergenic region and cea gene of EcN.

Another object of the present invention is to provide a composition for transforming EcN comprising:
the vector;
a guide RNA (gRNA) comprising a sequence complementary to a portion of the exo gene, exo/cea intergenic region, and cea gene sequence of Escherichia coli Nissle 1917 (EcN); and
a nucleic acid encoding a Cas protein.

Another object of the present invention is to provide a method for producing an EcN strain expressing TARS of Akkermansia muciniphila (AmTARS), comprising treating Escherichia coli Nissle 1917 (EcN) with the composition.

Another object of the present invention is to provide the use of the transformed EcN strain for manufacturing a composition for treating inflammatory diseases.

Another object of the present invention is to provide a method for treating inflammatory diseases comprising administering an effective amount of a composition comprising the transformed EcN strain as an active ingredient to a subject in need thereof.

Another object of the present invention is to provide a composition for preventing, improving, or treating inflammatory diseases consisting of the transformed EcN strain.

Another object of the present invention is to provide a composition for preventing, improving, or treating inflammatory diseases consisting essentially of the transformed EcN strain.

### Means for Solving the Problem

To achieve the aforementioned objectives of the present invention, the present invention provides an Escherichia coli Nissle 1917 (EcN) strain transformed with a vector comprising a nucleic acid encoding TARS of Akkermansia muciniphila (AmTARS).

To achieve another objective of the present invention, the present invention provides a composition for preventing, improving, or treating inflammatory diseases, comprising the transformed EcN strain.

To achieve another objective of the present invention, the present invention provides a vector comprising a 5′-homology arm, a promoter, a ribosome binding site consisting of the nucleotide sequence of SEQ ID NO: 2 (AGGAGGT), a nucleic acid encoding TARS of Akkermansia muciniphila (AmTARS), and a 3′-homology arm,
wherein the 5′-homology arm comprises a nucleotide sequence corresponding to a portion of the exo gene and exo/cea intergenic region of Escherichia coli Nissle 1917 (EcN),
and the 3′-homology arm comprises a nucleotide sequence corresponding to a portion of the exo/cea intergenic region and cea gene of EcN.

To achieve another object of the present invention, the present invention provides a composition for transforming EcN, comprising: the vector;
a guide RNA (gRNA) comprising a sequence complementary to a portion of the exo gene, exo/cea intergenic region, and cea gene sequence of Escherichia coli Nissle 1917 (EcN); and
a nucleic acid encoding a Cas protein.

To achieve another object of the present invention, the present invention provides a method for producing an EcN expressing TARS of Akkermansia muciniphila (AmTARS), comprising treating Escherichia coli Nissle 1917 (EcN) with the composition.

To achieve another object of the present invention, the present invention provides the use of the transformed EcN strain for manufacturing a composition for treating inflammatory diseases.

To achieve another object of the present invention, the present invention provides a method for treating inflammatory diseases, comprising administering an effective amount of a composition comprising the transformed EcN strain as an active ingredient to a subject in need thereof.

To achieve another object of the present invention, the present invention provides a composition for preventing, improving, or treating inflammatory diseases, consisting of the transformed EcN strain.

To achieve another object of the present invention, the present invention provides a composition for preventing, improving, or treating inflammatory diseases, essentially consisting of the transformed EcN strain.

In the present specification, the term “comprising” is used in the same sense as “including” or “characterized by” and does not exclude additional components or steps of methods that are not specifically mentioned in the composition or method according to the present invention. Furthermore, the term “consisting of” means excluding additional elements, steps, or components not specifically described. The term “essentially consisting of” means that the composition or method may include substances or steps that do not substantially affect the essential characteristics of the described substances or steps within the scope of the composition or method.

The term “effective amount” in the present invention refers to an amount that, when administered to a subject, exhibits an effect of improving, treating, detecting, diagnosing, or suppressing or reducing inflammatory diseases or inflammatory conditions. The term “subject” may include animals, preferably mammals, particularly humans, and may also include cells, tissues, or organs derived from animals. The subject may be a patient in need of the aforementioned effects.

In the present invention, the terms “nucleic acid,” “nucleic acid molecule,” “nucleic acid oligomer,” “oligonucleotide,” “nucleic acid sequence,” “nucleic acid fragment,” and “polynucleotide” are used interchangeably and are intended to include, but are not limited to, polymeric forms of nucleotides linked together, deoxyribonucleotides or ribonucleotides, or their analogs, derivatives, or modifications, which may have various lengths. Different polynucleotides may have different three-dimensional structures and may perform various known or unknown functions. Non-limiting examples of polynucleotides include genes, gene fragments, exons, introns, intergenic DNA (including but not limited to heterochromatic DNA), messenger RNA (mRNA), transfer RNA, ribosomal RNA, ribozymes, cDNA, sgRNA, guide RNA, tracrRNA, recombinant polynucleotides, branched polynucleotides, plasmids, vectors, isolated DNA sequences, isolated RNA sequences, PCR products, nucleic acid probes, and primers.

Polynucleotides useful in the methods disclosed herein may include natural nucleic acid sequences and their variants, artificial nucleic acid sequences, or combinations of the above sequences.

In the present invention, the terms “polypeptide,” “peptide,” and “protein” are used interchangeably to refer to polymers of amino acid residues, wherein the polymer may be conjugated to moieties that are not composed of amino acids. The above term applies not only to amino acid polymers, which are artificial chemical mimics of naturally occurring amino acids corresponding to one or more amino acid residues, but also to naturally occurring amino acid polymers and non-naturally occurring amino acid polymers. A “fusion protein” refers to a chimeric protein that encodes two or more distinct protein sequences recombinantly expressed as a single moiety.

With respect to amino acid sequences, those skilled in the art will recognize that individual substitutions, deletions, or additions to nucleic acid, peptide, polypeptide, or protein sequences that change, add, or delete a single amino acid or a small proportion of encoded amino acids may result in “conservatively modified variants” where the amino acid is substituted with a chemically similar amino acid. Conservatively substituted variants that provide functionally similar amino acids are well known in the art. These conservatively modified variants are included in the polymorphic variants, interspecies homologs, and alleles disclosed herein and do not exclude them.

The following eight groups each comprise amino acids that are conservative substitutions for one another: 1. Alanine (A), Glycine (G); 2. Aspartic acid (D), Glutamic acid (E); 3. Asparagine (N), Glutamine (Q); 4. Arginine (R), Lysine (K); 5. Isoleucine (I), Leucine (L), Methionine (M), Valine (V); 6. Phenylalanine (F), Tyrosine (Y), Tryptophan (W); 7. Serine(S), Threonine (T); and 8. Cysteine (C), Methionine (M).

For specific proteins described herein, the named protein comprises any of the naturally occurring forms, natural or engineered variants, or homologs of the protein that maintain the activity of the protein (e.g., at least 50%, 80%, 90%, 95%, 96%, 97%, 98%, 99%, or 100% activity compared to the natural protein). In embodiments, the variant or homolog has at least 90%, 95%, 96%, 97%, 98%, 99%, or 100% amino acid sequence identity to the naturally occurring form over the entire sequence or a portion of the sequence (e.g., portions of 50, 100, 150, or 200 consecutive amino acid).

In the present invention, the term “homology arm” refers to sequences homologous to endogenous chromosomal nucleic acid sequences located on either side of the desired genetic modification/replacement/insertion site, for example, in the present invention, on the 5′ and 3′ directions of the site where the nucleic acid encoding AmTARS is to be inserted. Homologous nucleic acid sequences may be two or more nucleic acid sequences that are identical or similar enough to hybridize with each other or undergo inter-molecular exchange. Due to the homology between the homology arm and the corresponding endogenous sequence, the homology arm guides the repair template to be inserted at a specific chromosomal location in the genome, i.e., between the endogenous sequences corresponding to the 5′-homology arm and the 3′-homology arm.

In the present invention, the term “homology-directed repair” or “HDR” refers to an intracellular mechanism for correctly and accurately repairing double-strand DNA breaks using a homology template for guided repair. The mechanism underlying HDR is homologous recombination (HR). The term “homologous recombination” or “HR” refers to a genetic process in which nucleotide sequences are exchanged between two similar molecules of DNA.

In the present invention, the term “CRISPR” or “clustered regularly interspaced short palindromic repeats” is used in its ordinary general sense and refers to a genetic element used by bacteria as a form of acquired immunity to protect against viruses. CRISPR includes short sequences derived from viral genomes and integrated into bacterial genomes. Cas (CRISPR-associated protein) proteins process these sequences and cleave matching viral DNA sequences. Thus, CRISPR sequences serve to guide Cas to recognize and cleave DNA that is at least partially complementary to the CRISPR sequence. By introducing plasmids containing Cas genes and specifically designed CRISPRs into eukaryotic cells, the eukaryotic genome can be cleaved at any desired location. Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In the present invention, the term “Cas9” or “CRISPR-associated protein 9” is used in its ordinary general sense and refers to an enzyme that recognizes and cleaves specific strands of DNA that are at least partially complementary to the CRISPR sequence using the CRISPR sequence as a guide. The Cas9 enzyme forms the basis of the technology known as CRISPR-Cas9, which can be used to edit genes within an organism in conjunction with CRISPR sequences. “Cas9,” “Csn1,” or “Cas9 protein” comprises any recombinant or naturally occurring form of Cas9 endonuclease or its variants or homologs that maintain Cas9 endonuclease activity (e.g., at least 50%, 80%, 90%, 95%, 96%, 97%, 98%, 99%, or 100% activity compared to Cas9). In one aspect, the variant or homolog has at least 90%, 95%, 96%, 97%, 98%, 99%, or 100% amino acid sequence identity to the naturally occurring Cas9 protein over the entire sequence or a portion of the sequence (e.g., 50, 100, 150, or 200 consecutive amino acid portions). In one aspect, the Cas9 protein is substantially identical to the protein identified by UniProt reference number Q99ZW2 or a variant or homolog thereof with substantial identity. In one aspect, the Cas9 protein has at least 75%, 80%, 85%, 90%, or 95% sequence identity to the amino acid sequence of the protein identified by UniProt reference number Q99ZW2. In one aspect, the Cas9 enzyme may be a mutant derived from Cas9 of S. pneumoniae, S. pyogenes, or S. thermophilus, or from these organisms.

In the present invention, the term “CRISPR-associated endonuclease Cas12a,” “Cas12a,” “Cas12,” or “Cas12 protein” comprises any recombinant or naturally occurring form of Cas12 endonuclease or its variants or homologs that maintain Cas12 endonuclease activity (e.g., at least 50%, 80%, 90%, 95%, 96%, 97%, 98%, 99%, or 100% activity compared to Cas12). In one aspect, the variant or homolog has at least 90%, 95%, 96%, 97%, 98%, 99%, or 100% amino acid sequence identity to the naturally occurring Cas12 protein over the entire sequence or a portion of the sequence (e.g., 50, 100, 150, or 200 consecutive amino acid portions). In embodiments, the Cas12 protein is substantially identical to the protein identified by UniProt reference number A0Q7Q2 or a variant or homolog thereof with substantial identity.

In the present invention, the term “CRISPR-associated endoribonuclease Cas13a,” “Cas13a,” “Cas13,” or “Cas13 protein” comprises any recombinant or naturally occurring form of Cas13 endoribonuclease or its variants or homologs that maintain Cas13 endoribonuclease activity (e.g., at least 50%, 80%, 90%, 95%, 96%, 97%, 98%, 99%, or 100% activity compared to Cas13). In one aspect, the variant or homolog has at least 90%, 95%, 96%, 97%, 98%, 99%, or 100% amino acid sequence identity to the naturally occurring Cas13 protein over the entire sequence or a portion of the sequence (e.g., 50, 100, 150, or 200 consecutive amino acid portions). In embodiments, the Cas13 protein is substantially identical to the protein identified by UniProt reference number P0DPB8 or a variant or homolog thereof with substantial identity.

In the present invention, the term “guide RNA” or “gRNA” refers to an RNA sequence that has sufficient complementarity to a target polynucleotide sequence to hybridize with the target sequence and direct sequence-specific binding of the CRISPR complex to the target sequence. For example, gRNA can guide Cas to the target polynucleotide. In embodiments, the gRNA comprises crRNA and tracrRNA. For example, the gRNA may comprise crRNA and tracrRNA hybridized by base pairing. Thus, in embodiments, the two RNAs may be separately encoded by crRNA and tracrRNA as two RNA molecules, which then form an RNA/RNA complex due to complementary base pairing between crRNA and tracrRNA. In one aspect, the degree of complementarity between the guide RNA sequence and its corresponding target sequence is about 50%, 60%, 75%, 80%, 85%, 90%, 95%, 97.5%, 99%, or more when optimally aligned using a suitable alignment algorithm.

The terms “sgRNA,” “single guide RNA,” and “single guide RNA sequence” are used interchangeably and refer to an RNA sequence comprising crRNA and tracrRNA sequences. For example, the sgRNA may be a single RNA sequence comprising crRNA and tracrRNA. For example, the sgRNA may be a fusion sequence comprising crRNA and tracrRNA. In embodiments, the sgRNA is synthesized in vitro. In embodiments, the sgRNA is produced in vivo from a DNA sequence encoding the sgRNA.

In the present invention, the term “crRNA” refers to CRISPR RNA, a short guide RNA comprising a unique single repeat-spacer unit. In bacterial cells, crRNA interferes with the invasion of homologous foreign genomes by targeting foreign DNA. Thus, short mature crRNA is a key element in the interference stage of the immune pathway. crRNA comprises a nucleotide sequence that is at least partially complementary to the target DNA. Thus, crRNA directs target sequence recognition and enables specificity for the CRISPR gene-editing mechanism. In embodiments, the crRNA may be provided as pre-crRNA. The pre-crRNA may form an RNA duplex by forming a complex with a region at least partially complementary to tracrRNA. The pre-crRNA may be cleaved by a ribonuclease (e.g., RNase III) to generate a crRNA/tracrRNA hybrid. The hybrid serves as a guide for the endonuclease Cas9 to cleave the invading nucleic acid.

In the present invention, the term “tracrRNA” or “trans-activating crRNA” refers to a small trans-encoded RNA. TracrRNA is at least partially complementary to crRNA and base pairs with it to form an RNA duplex. In embodiments, the tracrRNA forms an RNA duplex with pre-crRNA. TracrRNA can function as a binding scaffold for Cas (e.g., Cas9) by non-covalently binding to Cas. In embodiments, recognition and binding of tracrRNA by Cas result in the formation of a Cas9/tracrRNA/crRNA complex.

In the present invention, the terms “electroporation,” “electropermeabilization,” and “electrotransfer” are used in their ordinary general sense and refer to a technique in which an electric field is applied to cells to increase the permeability of the cell membrane, allowing the introduction of chemicals, drugs, proteins, nucleic acids, or combinations thereof into the cells. Subsequently, the cells must be handled carefully until they have the opportunity to divide. This process is approximately 10 times more effective than chemical transformation. Thus, the term “electroporation enhancer” refers to a compound or composition that improves the delivery of chemicals, drug compounds, proteins, or nucleic acids into cells, improves the efficiency of genetic transformation of cells, and/or increases the level of cell survival after transfection. In embodiments, the electroporation enhancer improves the delivery of chemicals, drug compounds, proteins, or nucleic acids into cells. In embodiments, the electroporation enhancer increases the efficiency of genetic transformation in cells. In embodiments, the electroporation enhancer increases the efficiency of genetic transformation in cells compared to the efficiency of genetic transformation in the absence of the electroporation enhancer. In embodiments, the electroporation enhancer increases the level of cell survival after transfection. In embodiments, the electroporation enhancer increases the level of cell survival after transfection compared to cell survival after transfection in the absence of the electroporation enhancer.

In the present invention, the term “transfection” is used in its ordinary general sense and refers to the process of intentionally introducing naked or purified nucleic acids into eukaryotic cells.

Hereinafter, the present invention will be described in detail.

The present invention provides an Escherichia coli Nissle 1917 (EcN) strain transformed with a vector comprising a nucleic acid encoding TARS of Akkermansia muciniphila (AmTARS).

In the present invention, the strain may be characterized by expressing AmTARS.

In the present invention, the term “expression” means producing and/or secreting AmTARS.

In one aspect of the present invention, the vector may be characterized by comprising a 5′-homology arm, a promoter, a ribosome binding site consisting of the nucleotide sequence of SEQ ID NO: 2 (AGGAGGT), a nucleic acid encoding TARS of Akkermansia muciniphila (AmTARS), and a 3′-homology arm.

In one aspect of the present invention, the promoter may be selected from the group consisting of lac promoter, trp promoter, Tac promoter, lacUV5 promoter, lpp promoter, pLλ promoter, pRλ promoter, rac5 promoter, amp promoter, recA promoter, SP6 promoter, trp promoter, T7 promoter, pBAD promoter, Tet promoter, trc promoter, pepT promoter, sulA promoter, pol 11 (dinA) promoter, ruv promoter, uvrA promoter, uvrB promoter, uvrD promoter, umuDC promoter, lexA promoter, cea promoter, caa promoter, recN promoter, pagC promoter, hip promoter, ansB promoter, pflE promoter, and tetR/A promoter, but is not limited thereto. Preferably, the promoter may be selected from the group consisting of lac promoter, trp promoter, and Tac promoter, and most preferably, it may be a lac promoter. According to one embodiment of the present invention, the promoter may consist of the nucleotide sequence of SEQ ID NO: 6 or may exhibit 70%, 75%, 80%, 85%, 90%, 91%, 92%, 93%, 94%, 95%, 96%, 97%, 98%, or 99% sequence homology thereto.

[SEQ ID NO: 6]
TTTACACTTTATGCTTCCGGCTCGTATGTTG

In the present invention, the term “TARS (threonyl-tRNA synthetase)” refers to one of the AARS (Aminoacyl-tRNA synthetases), which are enzymes that link amino acids to transfer RNA (tRNA) during the initial stage of protein synthesis. Among human-derived AARS proteins, some are known to be secreted from cells and perform various functions. For example, under specific conditions, human YARS (tyrosyl-tRNA synthetase) is secreted outside the cell and functions like a cytokine, and human KARS (lysyl-tRNA synthetase) is known to stimulate immune cells and promote inflammatory responses. Specifically, human TARS (threonyl-tRNA synthetase) has been reported to induce angiogenesis through inflammatory signaling when secreted outside the cell and to increase the synthesis of MUC1 (Mucin 1), a major protein of mucin containing a large amount of threonine, in pancreatic cancer cells. Additionally, it has been reported to function as a key component of the vertebrate-specific protein synthesis initiation complex required for the synthesis of proteins encoded by genes essential for the development of blood vessels and the nervous system during the evolutionary transition from invertebrates to vertebrates.

In the present invention, the TARS of Akkermansia muciniphila (AmTARS) may, for example, comprise the amino acid sequence of SEQ ID NO: 7, but is not limited thereto. A protein comprising the amino acid sequence of SEQ ID NO: 7 may be used interchangeably with a protein having the amino acid sequence of SEQ ID NO: 7 or a protein consisting of the amino acid sequence of SEQ ID NO: 7.

[SEQ ID NO: 7]
MSEHKERKTLEERQQMSDLERLRHSCAHVLATAICRLWPDAQLAGGPAV
DNGFYYDVELDHRISTEDFERIEEEMKKVVKENQVFQKEVISRADAMKM
AESGELGALGPRSEPSRFKIDLLNDIPEDEEISLYRNGDFTDLCAGPHV
GRTGNCKAFKIMSVASAFYKGDKNRPMLQRIYGTCFPNRTQLDEHLERL
EEARRRDHRKLGRELGLFCIDESVGQGLILWKPKGALIRRSLQDFITEE
LDKLGYSQVYTPNIGKLDLYRTSGHFPYYQESQYAPIPERDAMEKLSQE
GASCAELFNGLATGTIEGYMLKPMNCPHHIKIYANDAHSYRDLPVRLAE
FGTVYRWEQSGELGGMTRVRGFTQDDAHIFCTPDQLAGEIRQCLGIVKT
IFGTLGMTDYRVRLSMRDPESDKYVGSPENWDKAEQALREAAEWLGADY
SEEAGEAAFYGPKIDFIVRDAIGREWQLGTVQVDYNLPERFDLHYTGAD
NKPHRPVMVHRAPFGSMERFTGLLIEHFEGKFPTWLSPEQVRVLPISDK
VTDVAAAHRTALAARGVRVTVDETPDKIGAKIRNARLDRVPYMLVLGQR
EAEDGTVSVRHRDKGDLGAMPFEQFADLVAREIAERHISPVI

In the present invention, SEQ ID NO: 7 refers to an amino acid sequence having TARS activity. Specifically, SEQ ID NO: 7 may be a protein sequence having TARS activity encoded by a gene coding for TARS. For the purposes of the present invention, the protein may specifically be derived from the genus Akkermansia and more specifically from Akkermansia muciniphila, but is not limited thereto, and sequences having the same activity as the above amino acid sequence may be included without limitation. The amino acid sequence of SEQ ID NO: 7 can be obtained from a known database such as GenBank (GenBank: ACD05550.1). The TARS may be used interchangeably with AmTARS [TARS derived from Akkermansia muciniphila].

Additionally, in the present invention, although the protein having TARS activity is defined as a protein comprising the amino acid sequence of SEQ ID NO: 7, this does not exclude the addition of meaningless sequences before or after the amino acid sequence of SEQ ID NO: 7, naturally occurring mutations, or potential silent mutations. If a protein comprising the amino acid sequence of SEQ ID NO: 7 has the same or corresponding activity, it is apparent to those skilled in the art that it falls within the scope of the protein having TARS activity of the present invention.

For example, the protein having TARS activity of the present invention may be a protein consisting of the amino acid sequence of SEQ ID NO: 7 or an amino acid sequence having 70%, 80%, 85%, 90%, 95%, 96%, 97%, 98%, or 99% or more homology or identity thereto. Additionally, if an amino acid sequence that exhibits such homology or identity, exhibits corresponding efficacy to the above protein, a protein consisting of an amino acid sequence in which some sequences are deleted, modified, substituted, or added may also fall within the scope of the protein of the present invention.

That is, even if the present invention describes “a protein or polypeptide consisting of the amino acid sequence described by a specific SEQ ID NO,” it is apparent that if the polypeptide consisting of the amino acid sequence of the corresponding SEQ ID NO has the same or corresponding activity, a protein consisting of an amino acid sequence in which some sequences are deleted, modified, substituted, or added may also be used in the present invention. For example, “a polypeptide consisting of the amino acid sequence of SEQ ID NO: 7” may fall within the scope of “a polypeptide consisting of the amino acid sequence of SEQ ID NO: 7” if it has the same or corresponding activity.

In the present invention, the term “homology” or “identity” refers to the percentage of identity between two polynucleotide or polypeptide moieties. The homology between sequences from one moiety to another can be determined by known techniques in the art. For example, the homology between two polynucleotide molecules or two polypeptide molecules can be determined by directly aligning the sequence information using sequence alignment and readily available computer programs. Additionally, the homology between polynucleotides can be determined by hybridizing the polynucleotides under conditions that form stable double strands between homologous regions and then determining the size of the fragments degraded by single-strand-specific nucleases.

In the present invention, the nucleic acid encoding the TARS may be derived from the genus Akkermansia and more specifically from Akkermansia muciniphila (i.e., AmTARS), but is not particularly limited as long as it is a microorganism of the genus Akkermansia capable of expressing a gene coding for TARS. The gene coding for TARS may comprise a nucleotide sequence coding for the amino acid sequence of SEQ ID NO: 7 and, more specifically, may comprise the nucleotide sequence of SEQ ID NO: 1, but is not limited thereto.

[SEQ ID NO: 1]
TGGCGGAATTCGGCACGGTGTACCGTTGGGAACAGAGCGGAGAGCTGGG
CGGCATGACGCGCGTGCGCGGTTTCACGCAGGATGACGCCCATATCTTC
TGCACGCCGGACCAGCTTGCCGGGGAAATCCGCCAGTGCCTGGGCATCG
TGAAAACCATTTTCGGTACGCTGGGCATGACGGACTACCGCGTGCGCCT
TTCCATGCGCGACCCGGAAAGCGACAAATACGTGGGTTCTCCGGAAAAT
TGGGACAAAGCGGAACAGGCTCTGCGGGAAGCTGCGGAATGGCTGGGGG
CGGACTACAGCGAGGAAGCTGGGGAAGCCGCCTTCTACGGCCCCAAGAT
CGACTTCATCGTGCGCGACGCCATCGGCCGCGAATGGCAGCTGGGAACC
GTGCAGGTGGACTACAACCTGCCGGAACGCTTTGACCTCCATTACACCG
GAGCGGACAACAAGCCGCACCGGCCCGTGATGGTGCACCGCGCCCCCTT
CGGCTCCATGGAACGCTTCACGGGGCTTCTGATTGAACACTTTGAAGGC
AAATTCCCCACCTGGCTTTCCCCGGAACAGGTGCGCGTGCTTCCCATCT
CCGACAAGGTGACGGACGTGGCCGCCGCGCATCGAACCGCCCTGGCCGC
CAGAGGCGTGCGCGTCACGGTGGACGAAACGCCGGACAAAATAGGCGCC
AAAATCCGCAATGCCCGTCTGGACCGCGTTCCCTACATGCTGGTGCTCG
GCCAGCGGGAGGCGGAAGACGGCACCGTCTCCGTCCGCCACCGGGACAA
GGGGGATCTGGGCGCGATGCCCTTTGAGCAATTCGCGGACCTCGTAGCC
CGGGAAATCGCGGAGCGTCATATTTCTCCCGTGATTTGA

In the present invention, the term “polynucleotide” refers to a polymer of nucleotides in which nucleotide monomers are covalently linked in a long chain, as a DNA or RNA strand of a certain length or more, and more specifically refers to a polynucleotide fragment coding for the above variant.

Specifically, the polynucleotide of the present invention may undergo various modifications within the coding region without altering the amino acid sequence of the polypeptide, considering the degeneracy of codons or the preferred codons in the organism in which the polypeptide is to be expressed. Specifically, any polynucleotide sequence coding for TARS of the present invention may be included without limitation. In one aspect of the present invention, the exo gene may consist of the nucleotide sequence of SEQ ID NO: 3.

[SEQ ID NO: 3]
Tgaaaccagactgaagcccacggtcgtcatcgggacgcaggccgccgcc
gtgcatggtatcagtgagcaggcactctgcggagcaccatcatggactg
atgtggtgcggcaactgcgtcatgcaatcggggaccgaccagtaattat
ctttaatgcccggttcgacatccgcattctgaaaaagactgctgccgca
catagcgatccggctgactggctggaagaactgacggtatattgtgtga
tggagctggctgcaggatattatggagcctccaaccgctatggcactat
ttcactggcctgtgctgccagccagaccggactgaactgggaagggcag
gcacactcagcgatcgctgacgcacggatgacggcaggggggtaaacgc
tattgctgcatatcatctggaactgctgcaggaacaggcacggctgaaa
acctga
In one aspect of the present invention, the
exo/cea intergenic region may consist of the
nucleotide sequence of SEQ ID NO: 4.
[SEQ ID NO: 4]
Ctgcctggcctgtataccgcaatcatttacgttatccagacgtaaatat
atcaataaatagcaatcccccagatac
In one aspect of the present invention, the cea
gene may consist of the nucleotide sequence of
SEQ ID NO: 5.
[SEQ ID NO: 5]
Ctaatgtagttccagcaagcacggccgggcagcttgttctgcctgcgtt
ttcttcaattgagcagtagaccatttagctgtggcatgaatggctgcag
aactttcactgttgctacctccagttccaccaccgctgccagagccact
cccgtctggattatcattcaaaagagtaatgattacctgccccttatca
tcataaggaacaccatctttatagtacgctacagctgtttccat

In one aspect of the present invention, the vector may be utilized as a repair template for inserting a nucleic acid encoding AmTARS into the exo/cea intergenic region of the EcN strain genome.

In one aspect of the present invention, the vector may be in the form of linear DNA, and in one example, the linear DNA may be prepared by cutting the vector using a restriction enzyme or amplifying it by polymerase chain reaction (PCR). Additionally, the vector may be a recombinant vector or a DNA strand.

In one aspect of the present invention, the vector may be characterized by comprising a 5′-homology arm, a promoter, a ribosome binding site consisting of the nucleotide sequence of SEQ ID NO: 2 (AGGAGGT), a nucleic acid encoding TARS of Akkermansia muciniphila (AmTARS), and a 3′-homology arm.

In one aspect of the present invention, the homology arm (i.e., 5′- and/or 3′-homology arm) may comprise a nucleotide sequence corresponding to a portion of the exo gene and exo/cea intergenic region of Escherichia coli Nissle 1917 (EcN) for the 5′-homology arm, and a nucleotide sequence corresponding to a portion of the exo/cea intergenic region and cea gene of EcN for the 3′-homology arm, so that the nucleic acid encoding AmTARS can be inserted into the exo/cea intergenic region of the EcN strain genome.

The length of the homology arm may be appropriately adjusted as needed, for example, each may be 50 to 1200 bp, 50 to 1000 bp, 50 to 800 bp, 50 to 700 bp, 50 to 600 bp, 50 to 500 bp, 100 to 1200 bp, 100 to 1000 bp, 100 to 800 bp, 100 to 700 bp, 100 to 600 bp, 100 to 500 bp, 200 to 1200 bp, 200 to 1000 bp, 200 to 800 bp, 200 to 700 bp, 200 to 600 bp, 200 to 500 bp, 300 to 1200 bp, 300 to 1000 bp, 300 to 800 bp, 300 to 600 bp, or 300 to 500 bp, but is not limited thereto.

If the length of the homology arm is excessively short, the efficiency of homologous-directed repair (HDR) or homologous recombination (HR) may decrease, and if the homology arm is excessively long, the size of the vector introduced into the cell may become excessively large, reducing transformation efficiency.

The homology arm may be appropriately selected and designed by a person skilled in the art according to the target location (i.e., the exo/cea intergenic region of EcN in the present invention) where the target gene (i.e., the nucleic acid encoding AmTARS in the present invention) is to be introduced.

The homology arm attached to the 5′ end of the target gene may comprise a nucleic acid sequence in which the PAM (protospacer adjacent motif) sequence is replaced with another sequence. The PAM sequence is known to specify the location where the Cas9 protein (Cas restriction enzyme) cuts and generally has the sequence NGG. Here, each “N” nucleotide in the PAM sequence represents “A,” “T,” “G,” or “C.” In this specification, the PAM sequence may mean ‘NNGG,’ which includes one additional nucleotide at the 5′ end of the generally known ‘NGG’ sequence.

The nucleic acid sequence replacing the PAM sequence may be used without limitation as long as it is not the sequence ‘NNGG’ to prevent specific recognition by Cas9, and for example, the NNGG nucleic acid sequence may be replaced with NNAA, NNAT, NNAG, NNAC, NNTA, NNTT, NNTG, NNTC, NNCA, NNCT, NNCG, NNCC, NNGA, NNGT, or NNGC nucleic acid sequences.

If a homology arm in which the PAM sequence is not replaced is attached to the 5′ end of the target gene, there is a risk that the Cas9 protein may repeatedly recognize and cut the corresponding region while introducing the target gene multiple times.

The vector may comprise additional components necessary for protein expression in cells, in addition to the essential components of the target protein AmTARS.

For example, the additional components may include expression regulatory elements, selection elements, and the like.

The expression regulatory elements may include promoters, enhancers, polyadenylation signals, Kozak consensus sequences, ITR (inverted terminal repeat), LTR (long terminal repeat), terminators, internal ribosome entry sites (IRES), 2A self-cleaving peptides, and the like.

For example, the promoter may be lac promoter, trp promoter, Tac promoter, SV40 early promoter, LTR (mouse mammary tumor virus long terminal repeat) promoter, Ad MLP (adenovirus major late) promoter, HSV (herpes simplex virus) promoter, CMV (cytomegalovirus) promoter, RSV (rous sarcoma virus) promoter, U6 promoter, and the like.

For example, the 2A self-cleaving peptides may include T2A, P2A, E2A, F2A, and the like. The vector for differentiation factor expression may comprise one or more 2A self-cleaving peptides. In this case, the 2A self-cleaving peptides generate multiple proteins from the same transcript. Thus, the 2A self-cleaving peptide may be located between two or more different proteins intended to be expressed in the vector.

The selection elements may include fluorescent protein genes, tags, reporter genes, antibiotic resistance genes, and the like.

For example, the fluorescent protein genes may include GFP gene (GFP gene), YFP gene (YFP gene), RFP gene (RFP gene), or mCherry gene (mCherry gene), and the like.

For example, the tags may include histidine (His) tag, V5 tag, FLAG tag, influenza hemagglutinin (HA) tag, Myc tag, VSV-G tag, and thioredoxin (Trx) tag, and the like.

For example, the reporter genes may include glutathione-S-transferase (GST), horseradish peroxidase (HRP), chloramphenicol acetyltransferase (CAT), beta-galactosidase, beta-glucuronidase, and the like.

For example, the antibiotic resistance genes may include hygromycin resistant gene, neomycin resistant gene, kanamycin resistant gene, blasticidin resistant gene, zeocin resistant gene, and the like.

In one aspect of the present invention, the vector may comprise the nucleotide sequence of SEQ ID NO: 8.

The present invention also provides a pharmaceutical composition for preventing or treating inflammatory diseases comprising the EcN strain transformed according to the above method and expressing AmTARS as an active ingredient.

In the present invention, the inflammatory disease refers to a disease in which inflammation is the main lesion.

The inflammatory disease may include, for example, inflammatory bowel disease (IBD), edema, dermatitis, conjunctivitis, periodontitis, rhinitis, otitis media, pharyngitis, tonsillitis, pneumonia, gout, ankylosing spondylitis, gastritis, psoriatic arthritis, osteoarthritis, periarthritis of the shoulder, tendinitis, tenosynovitis, myositis, hepatitis, lymphangitis, felon, urinary tract infection, peritonitis, cystitis, nephritis, respiratory disease, and sepsis, and specifically may be inflammatory bowel disease, and more specifically may be one or more selected from the group consisting of colitis, ulcerative colitis, Crohn's disease, and Behcet's enteritis, but is not limited thereto.

The inflammatory disease of the present invention may include immune diseases, metabolic diseases, and infectious diseases accompanied by inflammation.

The “immune diseases accompanied by inflammation” may include, for example, autoimmune diseases, transplant rejection, graft-versus-host disease, and the like. The autoimmune diseases may specifically include atopy, allergy, rheumatoid arthritis, Hashimoto's thyroiditis, multiple sclerosis, endometriosis, psoriasis, asthma, hypothyroidism, hyperthyroidism, Behcet's disease, myasthenia gravis, Meniere's syndrome, Guillain-Barre syndrome, Sjogren's syndrome, vitiligo, and systemic sclerosis, and specifically may be inflammatory bowel disease, and more specifically may be one or more selected from the group consisting of atopy, allergy, rheumatoid arthritis, Hashimoto's thyroiditis, multiple sclerosis, endometriosis, psoriasis, hypothyroidism, hyperthyroidism, and asthma, but is not limited thereto.

Recently, research results have been reported that metabolic diseases are caused by increased expression of inflammatory factors. The metabolic diseases may be caused by or accompanied by inflammation. The “metabolic diseases accompanied by inflammation” may include obesity, diabetes, insulin resistance, lipid metabolism disorders, hypertriglyceridemia, increased free fatty acids, decreased high-density cholesterol, or hypertension, and various complications induced thereby, and specifically may be one or more selected from the group consisting of obesity and diabetes, but is not limited thereto.

The “infectious diseases accompanied by inflammation” refer to inflammatory diseases caused by the transmission or invasion of disease-causing pathogens such as viruses, bacteria, fungi, or parasites into animals or humans. The inflammatory diseases are as described above.

In the present invention, the transformed strain may increase the secretion of IL-10.

In the present invention, the term “prevention” refers to any act of suppressing or delaying the onset of inflammatory diseases through the administration of the composition of the present invention, and “treatment” refers to any act of improving or beneficially altering the symptoms caused by inflammatory diseases through the administration of the composition.

The “treatment” of the present invention comprehensively refers to improving the symptoms caused by the inflammatory diseases, which may include curing, substantially preventing, or improving the condition of the diseases, and may include alleviating, curing, or preventing one or more symptoms or most symptoms caused by the diseases, but is not limited thereto.

The pharmaceutical composition of the present invention may further comprise an appropriate carrier, excipient, or diluent conventionally used in the manufacture of pharmaceutical compositions.

The pharmaceutical composition may have a formulation selected from the group consisting of tablets, pills, powders, granules, capsules, liquid preparations, syrups, sterile aqueous solutions, non-aqueous solvents, suspensions, emulsions, freeze-dried preparations, and suppositories, and may be in various oral or parenteral formulations.

When formulated, it is prepared using diluents or excipients such as fillers, extenders, binders, wetting agents, disintegrants, and surfactants commonly used. Solid preparations for oral administration include tablets, pills, powders, granules, and capsules, and such solid preparations are prepared by mixing one or more compounds with at least one excipient, for example, starch, calcium carbonate, sucrose, or lactose, gelatin, etc. In addition to simple excipients, lubricants such as magnesium stearate and talc may also be used. Liquid preparations for oral administration include suspensions, liquid preparations, emulsions, and syrups, which may include various excipients, for example, wetting agents, sweeteners, flavoring agents, and preservatives, in addition to simple diluents such as water and liquid paraffin. Preparations for parenteral administration include sterile aqueous solutions, non-aqueous solvents, suspensions, emulsions, freeze-dried preparations, and suppositories. Non-aqueous solvents and suspending agents may include injectable esters such as propylene glycol, polyethylene glycol, vegetable oils such as olive oil, and ethyl oleate. Bases for suppositories may include witepsol, macrogol, tween 61, cacao butter, laurin butter, and glycerogelatin.

The composition of the present invention can be administered in a pharmaceutically effective amount.

In the present invention, the term “pharmaceutically effective amount” refers to an amount sufficient to treat a disease with a reasonable benefit/risk ratio applicable to medical treatment, and the effective dosage level can be determined based on factors such as the type and severity of the individual, age, gender, type of disease, activity of the drug, sensitivity to the drug, administration time, administration route and excretion rate, treatment period, concurrently used drugs, and other factors well known in the medical field. The composition of the present invention can be administered as a single therapeutic agent or in combination with other therapeutic agents and can be administered sequentially or simultaneously with conventional therapeutic agents. It can also be administered in single or multiple doses. Considering all these factors, it is important to administer an amount that achieves maximum effect with minimal side effects, which can be easily determined by those skilled in the art. The preferred dosage of the composition of the present invention varies depending on the condition and weight of the patient, the severity of the disease, the form of the drug, the route of administration, and the duration, but for a desirable effect, the TARS of Akkermansia muciniphila or its fragment of the present invention is preferably administered at 0.0001 to 100 μg/kg per day, more preferably at 0.001 to 100 μg/kg per day. Administration may be performed once a day or divided into several doses. The composition can be administered to various mammals such as mice, livestock, and humans through various routes, and the method of administration is not limited as long as it is a conventional method in the art, for example, oral, rectal, intravenous, intramuscular, subcutaneous, intrauterine, epidural, or intracerebrovascular injection.

In the present invention, the content of the composition is not significantly limited depending on the purpose or aspect of use, and for example, it may be 0.01 to 99% by weight, preferably 0.5 to 50% by weight, more preferably 1 to 30% by weight, based on the total weight of the composition. In addition, the pharmaceutical composition according to the present invention may further include additives such as pharmaceutically acceptable carriers, excipients, or diluents in addition to the active ingredient. The pharmaceutical composition of the present invention may include the strain prepared by the method of the present invention in an amount of 0.1 to 99.9% by weight and the carrier in an amount of 99.9% to 0.1% by weight.

Furthermore, the pharmaceutical composition of the present invention can be used not only as a medicine for humans but also in the form of veterinary medicine.

The present invention also provides a food composition for preventing or improving inflammatory diseases comprising the transformed strain as an active ingredient.

In the present invention, the term “improvement” refers to any act in which the symptoms of inflammatory diseases suspected in the subject or the symptoms of the subject are improved or benefited by using the composition.

The food composition of the present invention may include food-grade acceptable salts, which are useful as acid addition salts formed by food-grade acceptable free acids or metal salts formed by bases. As an example, inorganic acids and organic acids can be used as free acids. Inorganic acids may include hydrochloric acid, sulfuric acid, bromic acid, sulfurous acid, or phosphoric acid, and organic acids may include citric acid, acetic acid, maleic acid, fumaric acid, gluconic acid, and methanesulfonic acid. In addition, metal salts may include alkali metal salts or alkaline earth metal salts, sodium, potassium, or calcium salts. However, it is not limited thereto.

The food composition of the present invention may include forms such as pills, powders, granules, infusions, tablets, capsules, or liquid preparations, and foods to which the composition can be added may include, for example, various food types, such as beverages, gums, teas, vitamin complexes, and health supplements.

The components that can be included in the food composition of the present invention are not particularly limited to other components in addition to the essential component containing the active ingredient, and various herbal extracts, food auxiliary additives, or natural carbohydrates may be included as additional components, as in conventional foods. The content of the active ingredient in the food composition can be appropriately determined depending on the purpose of use (prevention, improvement, or therapeutic treatment). At this time, the content of the active ingredient included in the composition is not particularly limited, but it may include 0.0001% to 10% by weight, preferably 0.001% to 1% by weight, based on the total weight of the composition.

In addition, the food auxiliary additives may include conventional food auxiliary additives in the art, for example, flavoring agents, taste enhancers, coloring agents, fillers, stabilizers, etc.

Examples of the natural carbohydrates include monosaccharides, such as glucose and fructose; disaccharides, such as maltose and sucrose; and polysaccharides, such as dextrin and cyclodextrin, as well as sugar alcohols such as xylitol, sorbitol, and erythritol. In addition to the above, natural flavoring agents (e.g., rebaudioside A, glycyrrhizin, etc.) and synthetic flavoring agents (e.g., saccharin, aspartame, etc.) can be advantageously used as flavoring agents.

In addition, the food composition of the present invention may include various nutrients, vitamins, minerals (electrolytes), synthetic flavoring agents, and natural flavoring agents, such as flavoring agents, coloring agents, and fillers (cheese, chocolate, etc.), pectic acid and its salts, alginic acid and its salts, organic acids, protective colloid thickeners, pH adjusters, stabilizers, preservatives, glycerin, alcohol, carbonating agents used in carbonated beverages, etc. Furthermore, it may include pulp for the production of natural fruit juices, fruit juice beverages, and vegetable beverages. These components can be used independently or in combination.

In the present invention, the health supplement may include health functional foods and health foods.

The term “functional food” refers to a food for special health use (FoSHU), which is a term for food processed to efficiently exhibit biological regulatory functions in addition to providing nutrition, with high medical and therapeutic effects. Here, “functional” means obtaining useful effects for health purposes, such as regulating nutrients or physiological actions concerning the structure and function of the human body.

The food comprising the food composition of the present invention can be manufactured by methods commonly used in the art, and during the manufacturing process, raw materials and components commonly added in the art can be added. In addition, the formulation of the food can be manufactured without limitation as long as it is a formulation recognized as food. The food composition of the present invention can be manufactured in various forms of formulations and has the advantage of not causing side effects that may occur with long-term use of drugs, unlike general medicines, and can be highly portable.

The present invention also provides a vector comprising a 5′-homology arm, a promoter, a ribosome binding site consisting of the nucleotide sequence of SEQ ID NO: 2 (AGGAGGT), a nucleic acid encoding TARS of Akkermansia muciniphila (AmTARS), and a 3′-homology arm,
wherein the 5′-homology arm comprises a nucleotide sequence corresponding to a portion of the exo gene and exo/cea intergenic region of Escherichia coli Nissle 1917 (EcN), and the 3′-homology arm comprises a nucleotide sequence corresponding to a portion of the exo/cea intergenic region and cea gene of EcN.

In one aspect of the present invention, the vector is characterized by being utilized for transforming EcN to express AmTARS.

The present invention also provides the above-described vector;
a guide RNA (gRNA) comprising a sequence complementary to a portion of the exo gene, exo/cea intergenic region, and cea gene sequence of Escherichia coli Nissle 1917 (EcN); and
a nucleic acid encoding a Cas protein, as a composition for transforming EcN.

In the composition according to the present invention, the gRNA and Cas protein can cut specific regions of the exo gene, exo/cea intergenic region, and cea gene sequence of Escherichia coli Nissle 1917 (EcN), and the vector according to the present invention can be used as a repair template in the cut region to enable the production of EcN in which a gene encoding AmTARS is inserted through homology-directed repair (HDR) or homologous recombination (HR).

In one aspect of the present invention, the Cas protein may be selected from the group consisting of Cas9, Cas12, Cas13, and variants thereof.

The present invention also provides a method for producing EcN expressing TARS of Akkermansia muciniphila (AmTARS), comprising treating Escherichia coli Nissle 1917 (EcN) with the composition according to claim 9.

In one aspect of the present invention, the method may comprise (a) transfecting the Cas protein into EcN; and (b) transfecting the gRNA and the vector into EcN.

In one aspect of the present invention, the transfection in each step can be achieved by methods known in the art, such as heat shock, electroporation, chemical transfection, and lipid-based transfection.

In the method of the present invention, after steps (a) and (b), the vector can be inserted into the genome of EcN in the double-strand break (DSB) region formed by the Cas protein through homology-directed repair or homologous recombination.

If the double-strand break generated by the Cas protein is not repaired by recombinase, the strain dies and is eliminated during the selection process, allowing strain selection without antibiotic resistance genes.

The recombinase can be selected without limitation for the purpose of inserting the target gene into the genome of the target cell based on the homologous region, and for example, it may be RED recombinase. If the target gene is inserted into the genome of the target cell by the recombinase and the DSB is repaired, cell death does not occur.

To achieve another object of the present invention, the present invention provides the use of the transformed EcN strain for manufacturing a composition for treating inflammatory diseases.

To achieve another object of the present invention, the present invention provides a method for treating inflammatory diseases comprising administering an effective amount of a composition comprising the transformed EcN strain to a subject in need thereof.

To achieve another object of the present invention, the present invention provides a composition for preventing, improving, or treating inflammatory diseases, consisting of the transformed EcN strain.

To achieve another object of the present invention, the present invention provides a composition for preventing, improving, or treating inflammatory diseases, essentially consisting of the transformed EcN strain.

### Effect of the Invention

The vector according to the present invention and the composition for transformation comprising the same can be very effectively utilized in producing EcN that stably produces/secretes AmTARS, and the EcN strain produced using the same exhibits very excellent effects in the prevention, improvement, and treatment of inflammatory diseases.

### BRIEF DESCRIPTION OF THE DRAWINGS

FIG. 1 is a schematic diagram illustrating the process of producing probiotics *E. coli Nissle* [EcN(AmTARS-His)] that delivers the AmTARS-His protein.

FIGS. 2a to 2c show the results of confirming the expression of the AmTARS-His protein when a plasmid containing an expression cassette expressing the AmTARS-His protein was modified and introduced into the EcN strain.

FIG. 3 shows the results of confirming that the AmTARS-His expression gene was introduced into the genome of EcN(AmTARS).

FIG. 4 shows the results of confirming that the EcN(AmTARS-His) strain in the culture medium expresses and secretes the AmTARS-His protein.

FIG. 5 shows the results of confirming that the EcN(AmTARS-His) strain secretes the AmTARS-His protein and delivers it to intestinal immune cells when administered orally.

FIGS. 6a to 6h show the results of confirming the therapeutic efficacy of EcN(AmTARS-His) probiotics in a mouse model of inflammatory bowel disease.

FIG. 7 is a schematic diagram illustrating the process of producing probiotics *E. coli Nissle* [EcN(AmTARS)] that delivers the AmTARS protein with the His tag removed (including TCGCGGAGCGTCATATTTCTCCCGTGATTTGA of SEQ ID NO: 1).

FIG. 8 shows the results of comparing the expression and secretion of the AmTARS protein in the culture medium of the EcN(AmTARS) strain with that of EcN(AmTARS-His).

FIGS. 9a to 9h show the results of confirming the therapeutic efficacy of EcN(AmTARS) probiotics in a mouse model of inflammatory bowel disease.

### DETAILED DESCRIPTION OF THE INVENTION

Hereinafter, the present invention will be described in detail with reference to the following examples. However, the following examples are merely illustrative of the present invention, and the present invention is not limited thereto.

E. coli Nissle (EcN) AmTARS Gene Introduction and Transformation

Using the λ-Red-mediated CRISPR-Cas9 system, the Akkermansia muciniphila threonyl-tRNA synthetase (AmTARS) gene was introduced into the genome of the Escherichia coli Nissle 1917 (EcN) strain to construct EcN(AmTARS-His) or EcN(AmTARS), strains that express the AmTARS protein. To avoid affecting the expression of surrounding genes, the insertion site of the AmTARS gene targeted the exo/cea intergenic region sequence, and 20 nucleotides between the exo/cea sequences were introduced into the pTarget plasmid. The inserted gene included a 5′-homology arm comprising the exo gene sequence and the exo/cea intergenic sequence, and a 3′-homology arm comprising the exo/cea intergenic sequence and the cea gene sequence at the ends. Between these, a vector was constructed comprising a lac promoter (SEQ ID NO: 6), a ribosome binding site (SEQ ID NO: 2 (AGGAGGT)), and the AmTARS gene to be expressed (SEQ ID NO: 1) (SEQ ID NO: 8). To express the Cas9 protein in the EcN strain, the pCas plasmid was introduced, and subsequently, pTarget (sgRNA: SEQ ID NO: 9 (caatcatttacgttatccag)) and the inserted gene were co-transformed into the strain expressing Cas9. After culturing on LB agar plates containing kanamycin (pCas antibiotic marker) and spectinomycin (pTarget marker) at 30° C., strains with the inserted gene introduced into the exo/cea sequence were screened and secured.

### Immunoblot Analysis

A. muciniphila, EcN, EcN(AmTARS), and EcN(AmTARS-His) strains were pre-cultured, and 100 μl of each strain was inoculated into 50 ml of LB liquid medium and cultured at 37° C. for 3, 6, 12, and 24 hours. 10 ml of each culture was collected, centrifuged at 4000 rpm for 10 minutes, and the cells and supernatant were separated. The cells were resuspended in 1×PBS buffer, lysed by sonication, and centrifuged at 13000 rpm for 20 minutes. The culture supernatant was concentrated to a final volume of 100 μl using a 30K cut-off filter. For protein immunoanalysis in the samples, the samples were loaded onto a 7.5% SDS-PAGE gel and transferred to a PVDF membrane. To block nonspecific binding of antibodies, the membrane was reacted with 1×TBST (137 mM NaCl, 50 mM Tris-HCl, 0.1% Tween20) and 4% BSA solution for 1 hour and 30 minutes. The membrane was then reacted overnight at 4° C. with each antibody diluted in 1×TBST and 4% BSA solution according to its working concentration. The target protein was detected through a chemiluminescence signal.

### Mouse Management

The mouse line used in this study was 8-week-old C57BL/6. All mice were maintained at 22° C. with a 12-hour light/dark cycle and were used for experiments after genotyping analysis.

### Inflammatory Bowel Disease Mouse Model Preparation

To induce inflammatory bowel disease, 2% DSS (Dextran Sulfate Sodium) was administered in drinking water to 8-week-old mice for 10 days, and EcN, EcN(AmTARS), and EcN(AmTARS-His) strains were orally administered at a dose of 10⁷ cells/100 μl once daily.

### Enzyme-Linked Immunosorbent Assay (ELISA)

To obtain mouse plasma, the mice were anesthetized using isoflurane solution, and blood was collected from the heart after laparotomy. To obtain pure plasma, the blood was left at room temperature for 4 hours and then centrifuged at 1000×g for 20 minutes. To confirm the secretion of immune cytokines derived from mice, the mouse IL-10 ELISA set DUO from BD Biosciences was used.

### Flow Cytometry

Cells isolated from colon tissue (1×10⁶ cells) were washed once with staining buffer (PBS containing 1% FBS and 0.01% NaN₃, pH 7.4), and antibodies conjugated with fluorescent substances (F4/80: macrophages, CD11b: monocytes, CD206: M2 macrophages) were added to the samples and reacted at 4° C. for 20 minutes. The samples were then washed twice with staining buffer, and the expression of cell surface molecules was analyzed by flow cytometry.

### Tissue Staining (H&E and Immunofluorescence Staining)

Colon tissue was fixed in 10% paraformaldehyde, and paraffin sections were prepared. The sections were cut into 5 μm thickness and mounted on slides. Paraffin removal using xylene and hydration using ethanol were performed according to standard experimental methods. For H&E staining, the slides were reacted with hematoxylin for 1 minute and eosin for 30 seconds, followed by dehydration and observation under an optical microscope. For immunofluorescence staining, antigen retrieval was performed using antigen retrieval solution (0.1 M sodium citrate pH 6.0 and 0.05% Tween-20), and nonspecific antibody binding was blocked by reacting with 1×PBS solution containing 3% BSA for 1 hour. Antibodies conjugated with fluorescent substances (F4/80: macrophages, His: AmTARS) were reacted at room temperature for 2 hours, and residual antibodies were removed by washing three times with 1×PBS containing 0.1% Tween-20. The slides were then treated with mounting solution containing DAPI and observed under a fluorescence microscope.

### Measurement of Disease Activity and Epithelial Damage Scores

The disease activity score was calculated by summing the scores (0-4) for bloody stool, diarrhea, and weight loss. The epithelial damage score was calculated by assigning scores (0-4) based on the degree of damage to the colon tissue.

### Experimental Results

### Example 1. Production of Probiotics E. coli Nissle [EcN(AmTARS-His)] Expressing and Secreting A. muciniphila Threonyl-tRNA Synthetase (AmTARS) and

### Confirmation of AmTARS-His Protein Secretion

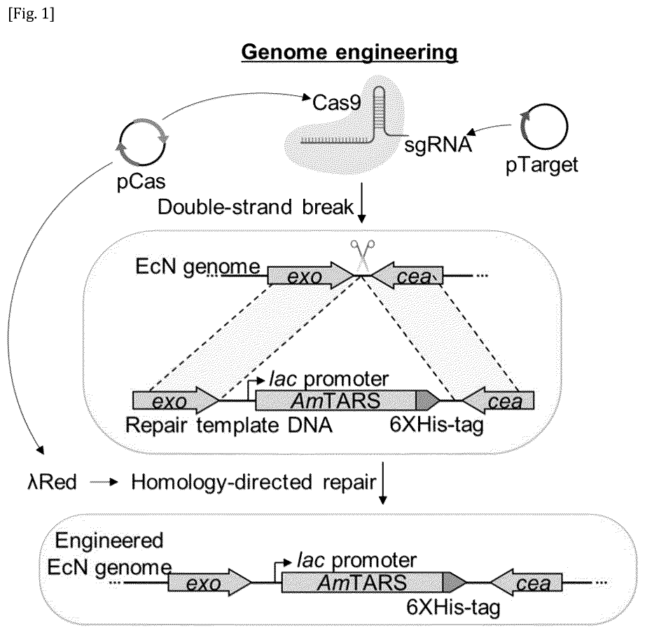

To produce the probiotics E. coli Nissle EcN(AmTARS-His) expressing and secreting the AmTARS-His protein derived from Akkermansia muciniphila, the λ-Red-mediated CRISPR-Cas9 system was used to insert the AmTARS gene into the strain genome. As shown in the schematic diagram in FIG. 1, engineering was performed by preparing the pCas plasmid expressing the Cas9 protein capable of recognizing and cutting specific gene sequences, the pTarget plasmid expressing sgRNA to assist in recognizing the gene insertion site, and the repair template containing the gene to be inserted.

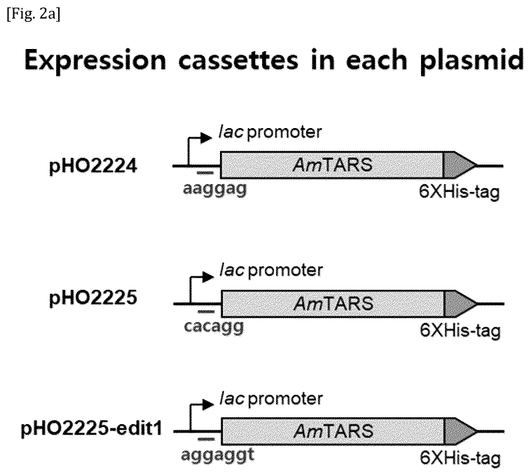

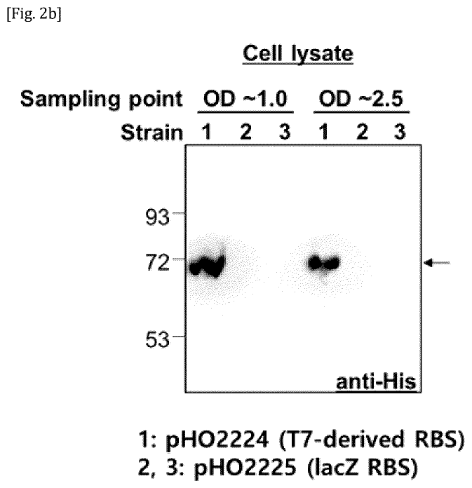

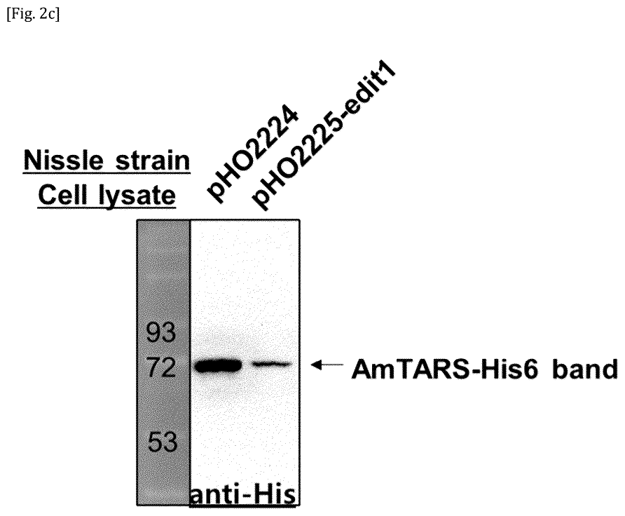

When attempting to introduce the AmTARS-His gene into the threonyl-tRNA synthetase gene (EcN_thrS) site in the EcN genome, the strain did not survive during transformation. Therefore, the AmTARS-His gene was inserted into another site in the genome to produce a strain that continuously expresses the AmTARS-His protein. First, it was tested whether the AmTARS-His protein was expressed when the gene was introduced in plasmid form into the strain. As shown in FIGS. 2a and 2b, when the plasmid (pHO2224) using the T7-derived RBS with the lac promoter was introduced into the strain, the AmTARS-His protein was confirmed to be expressed. However, a problem occurred in forming the repair template properly. Therefore, while keeping the promoter unchanged, a new plasmid pHO2225 was constructed using the native RBS of lacZ. Although the repair template could be constructed with pHO2225, the protein was not expressed in plasmid form when introduced into the strain. Thus, a new plasmid (pHO2225-edit1) was constructed by inducing a mutation in the RBS from the native lacZ RBS to the Shine-Dalgarno sequence. As shown in FIG. 2c, although the expression of the AmTARS-His protein with pHO2225-edit1 was weaker than with pHO2224, it was possible, and the repair template for genome insertion could be constructed.

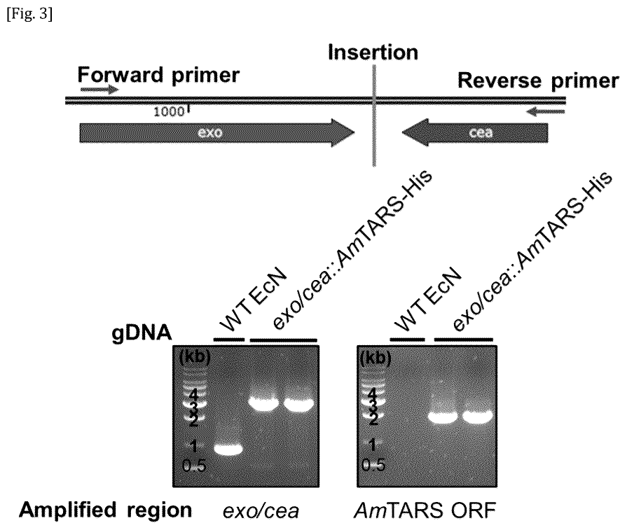

The expression cassette on pHO2225-edit1 was used as the repair template DNA and co-introduced with pTarget into the E. coli Nissle strain containing pCas via electroporation. The genomic DNA of the transformed strain was extracted, and strains containing the AmTARS-His gene in the exo/cea genome were screened. As shown in FIG. 3, the EcN(AmTARS-His) strain with the AmTARS-His expression cassette introduced into the exo/cea genome was secured.

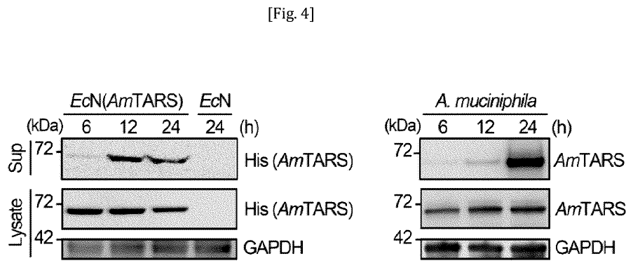

To confirm whether the AmTARS-His gene introduced into the genome was expressed and secreted, the EcN(AmTARS-His) strain was cultured in LB medium for 6, 12, and 24 hours. As shown in FIG. 4, immunoblot analysis confirmed that the AmTARS-His protein was expressed and secreted in the cell lysate and culture supernatant. The culture supernatant of EcN(AmTARS-His) was concentrated 40-fold for confirmation, while the culture supernatant of Akkermansia muciniphila was analyzed without concentration. It was confirmed that the secretion of the AmTARS protein by EcN(AmTARS-His) was approximately 40 times lower than that of Akkermansia.

### Example 2. Confirmation of AmTARS Secretion and Delivery to Intestinal Immune Cells Upon Oral Administration of EcN(AmTARS-His)

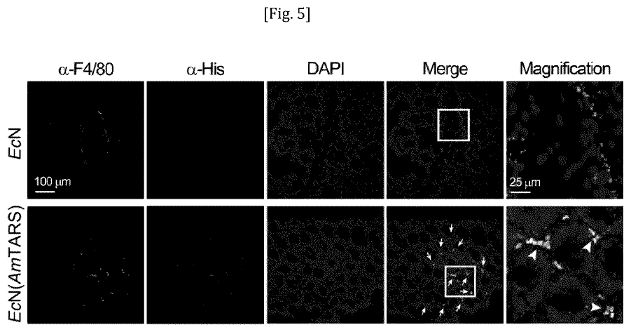

To confirm whether the EcN(AmTARS-His) strain expresses and secretes the AmTARS-His protein in the intestinal environment, EcN and EcN(AmTARS-His) strains were orally administered daily at a dose of 10⁷ cells/100 μl for 5 days. Colon tissue was then collected and analyzed by immunofluorescence. As shown in FIG. 5, the AmTARS-His protein present in the intestine was confirmed using a His antibody in the colon of mice administered with EcN(AmTARS-His). Additionally, co-localization with macrophages labeled with the F4/80 antibody was confirmed, demonstrating that the AmTARS-His protein was secreted into the intestinal environment and interacted with intestinal immune cells.

### Example 3. Verification of Anti-Inflammatory Efficacy of EcN(AmTARS-His) Strain in Inflammatory Bowel Disease Mouse Model

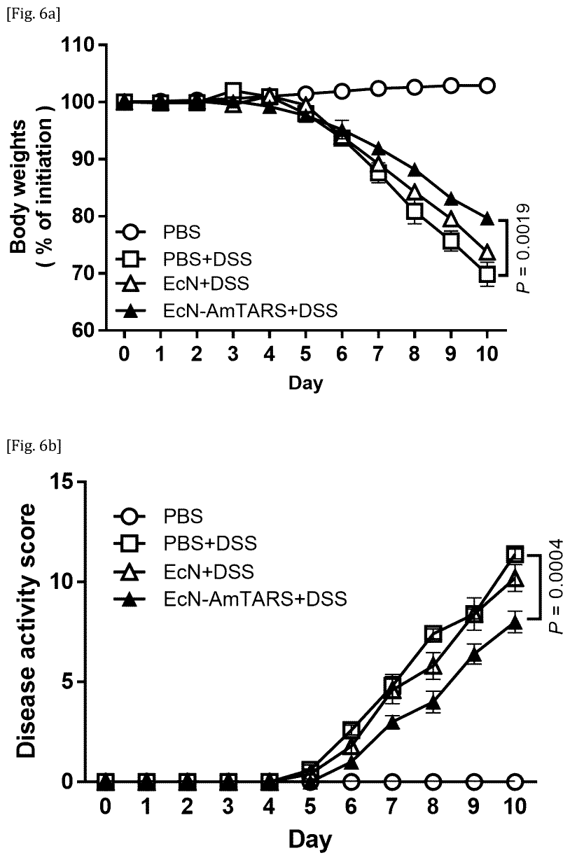

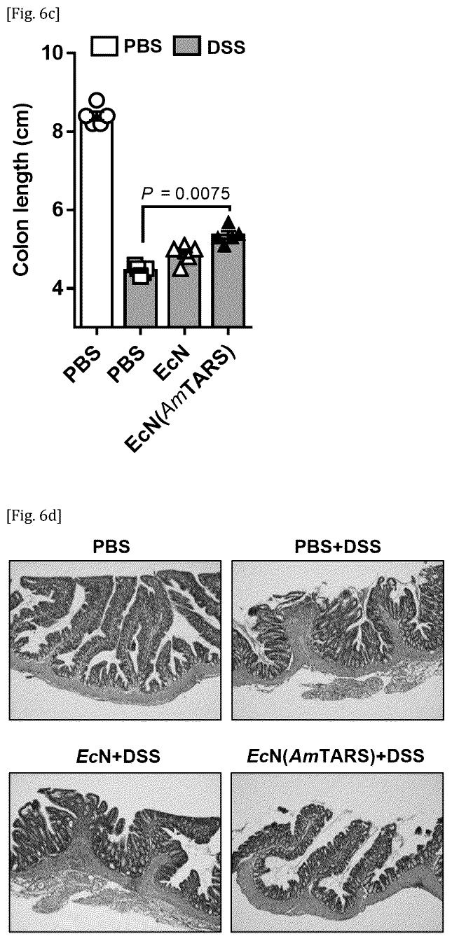

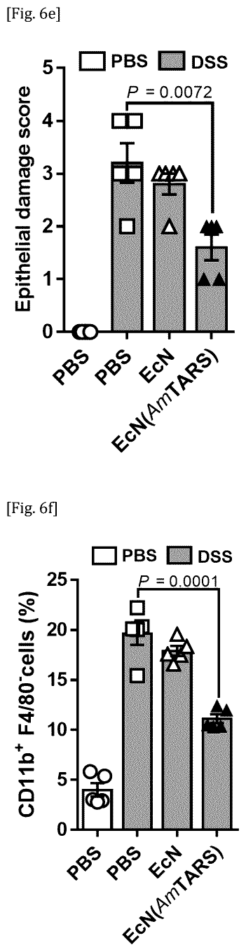

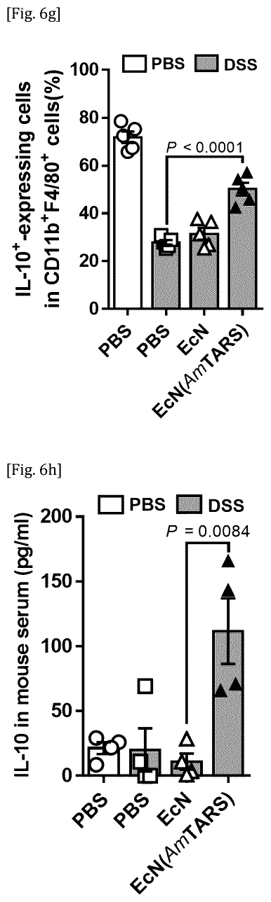

Based on the confirmation in Example 2 that the EcN(AmTARS-His) strain delivered via oral administration synthesizes and secretes the AmTARS-His protein in the intestinal environment, the anti-inflammatory efficacy of the EcN(AmTARS-His) strain was evaluated in an inflammatory bowel disease mouse model. Inflammatory bowel disease was induced in 8-week-old male C57BL/6 mice by administering 2% DSS (Dextran Sulfate Sodium) in drinking water. PBS group mice were orally administered 100 μl of 1×PBS daily, while EcN and EcN(AmTARS-His) groups were administered 10⁷ cells/100 μl daily. As shown in FIGS. 6a to 6h, the administration of EcN(AmTARS-His) alleviated inflammation, as evidenced by reduced disease activity scores, colon length reduction, and tissue damage. Flow cytometry analysis of intestinal immune cells revealed that the proportion of infiltrated inflammatory monocytes (CD11b⁺F4/80⁻) in the intestine was significantly reduced in the EcN(AmTARS-His) group compared to the PBS control group, while the proportion of M2 macrophages capable of secreting IL-10 (CD11b⁺F4/80⁺CD206⁺) increased. The concentration of IL-10 in mouse plasma was significantly increased in the EcN(AmTARS-His) group compared to the EcN group, confirming that the AmTARS-His protein secreted by the EcN(AmTARS-His) strain interacted with intestinal immune cells to exhibit anti-inflammatory efficacy.

### Example 4. Production of EcN(AmTARS) Strain Expressing AmTARS Protein without His Tag and Confirmation of AmTARS Protein Secretion

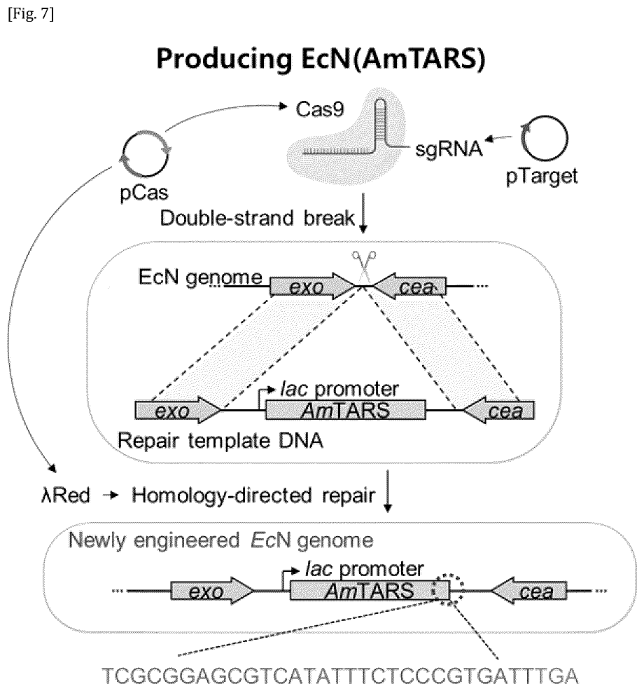

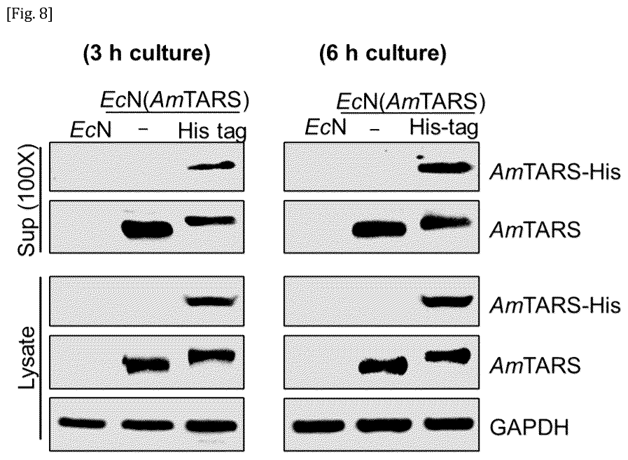

Since the engineered EcN(AmTARS-His) strain was confirmed in Examples 2, 3, and 4 to secrete the AmTARS-His protein in the intestinal environment and exhibit anti-inflammatory activity, a strain expressing the AmTARS protein without the His tag was constructed. As shown in FIG. 7, the same method used to construct the EcN(AmTARS-His) strain expressing and secreting AmTARS with a His tag at the C-terminus in Example 1 was applied. An expression cassette was used as the repair template, in which the 18-nucleotide sequence expressing the His tag (CACCACCACCACCACCA) was removed, and a stop codon (TAG) was inserted. To confirm the expression and secretion of the AmTARS protein by the EcN(AmTARS) strain expressing AmTARS without the His tag, the EcN(AmTARS) strain was cultured in LB medium for 3 and 6 hours, and the cells and culture supernatant were separated for immunoblot analysis, as performed in Example 1. As shown in FIG. 8, both EcN(AmTARS) and EcN(AmTARS-His) strains were confirmed to express and secrete the AmTARS protein using an AmTARS-specific antibody. When using a His tag antibody, AmTARS-His was detected only in the lysate and supernatant derived from the EcN(AmTARS-His) strain.

### Example 5. Verification of Anti-Inflammatory Efficacy of EcN(AmTARS) Strain in Inflammatory Bowel Disease Mouse Model

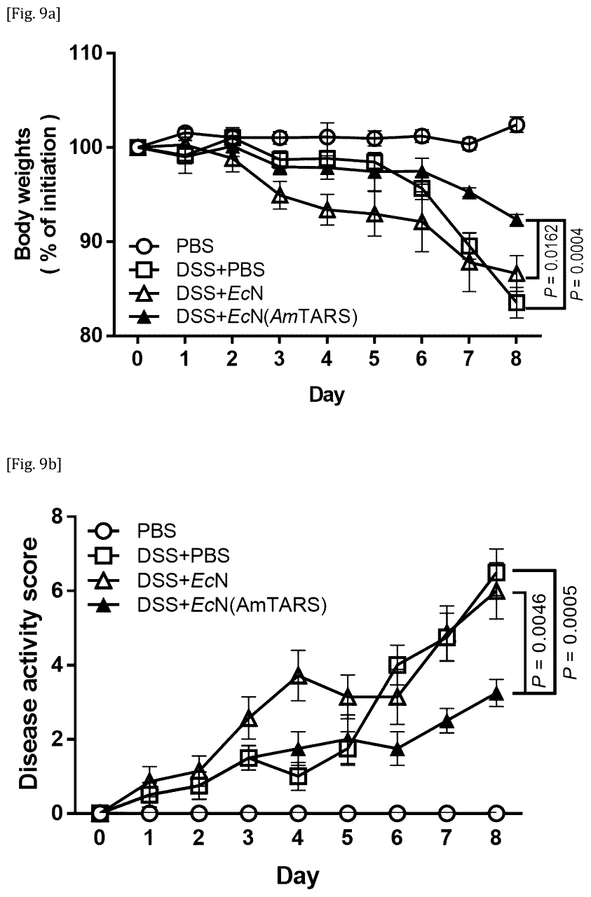

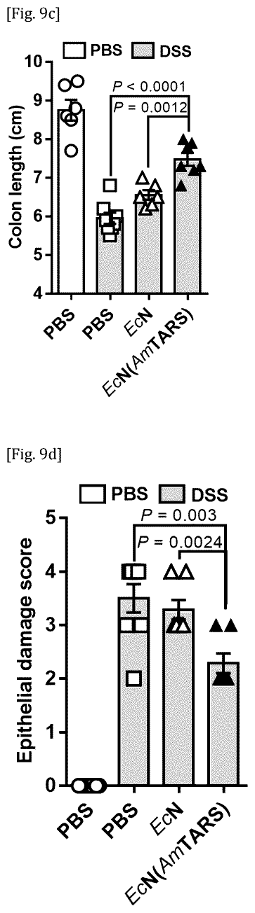

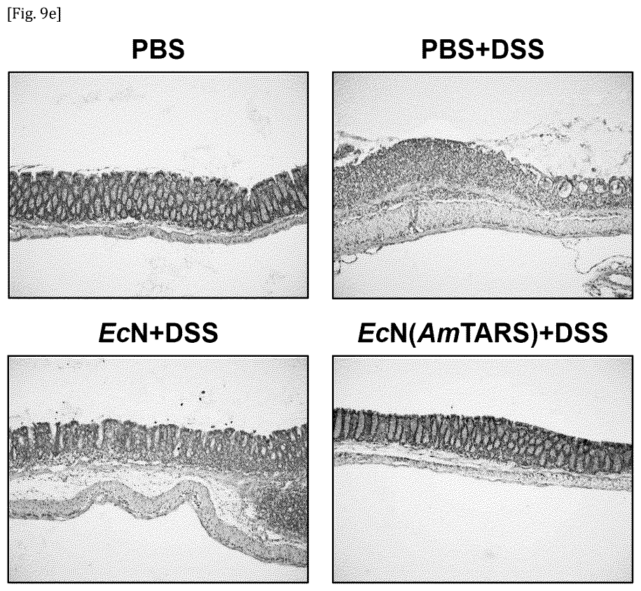

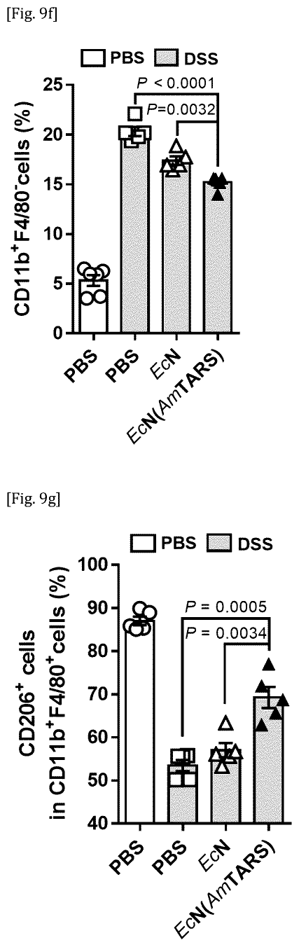

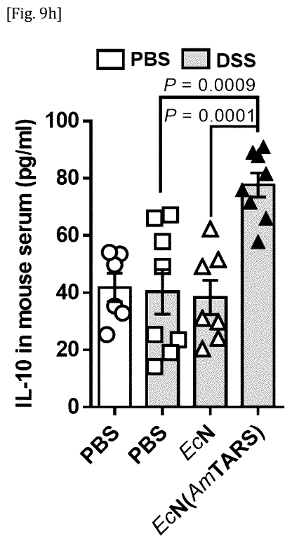

To evaluate the anti-inflammatory efficacy of the EcN(AmTARS) strain expressing the AmTARS protein, the same experiment as in Example 3 was conducted. Inflammatory bowel disease was induced in 8-week-old male C57BL/6 mice by administering 2% DSS (Dextran Sulfate Sodium) in drinking water. PBS group mice were orally administered 100 μl of 1×PBS daily, while EcN and EcN(AmTARS) groups were administered 10⁷ cells/100 μl daily. As shown in FIGS. 9a to 9h, the EcN(AmTARS) treatment group showed alleviation of disease activity scores, colon length reduction, and colon tissue damage compared to the control groups (PBS and EcN). Analysis of intestinal immune cells also revealed that the infiltration rate of intestinal monocytes (CD11b⁺F4/80⁻) was reduced compared to the two control groups, while the proportion of M2 macrophages (CD11b⁺F4/80⁺CD206⁺) increased. The amount of anti-inflammatory cytokine IL-10 in mouse plasma was also increased in the EcN(AmTARS) treatment group.

Through this, it was confirmed that the probiotics E. coli Nissle strain EcN(AmTARS), capable of secreting the AmTARS protein into the intestine, also exhibited anti-inflammatory efficacy due to AmTARS.

### INDUSTRIAL APPLICABILITY

The vector according to the present invention and the transformation composition comprising the same can be very effectively utilized for producing EcN that stably produces/secretes AmTARS, and the EcN produced using the same exhibits excellent effects in the prevention, improvement, and treatment of inflammatory diseases, thereby having high industrial applicability.

What is claimed is:

1. An Escherichia coli Nissle 1917 (EcN) strain transformed with a vector comprising a nucleic acid encoding TARS of Akkermansia muciniphila (AmTARS).

2. The strain according to claim 1, wherein the strain expresses AmTARS.

3. The strain according to claim 1, wherein the nucleic acid encoding AmTARS is introduced into the exo/cea intergenic region.

4. The strain according to claim 1, wherein the vector comprises a 5′-homology arm, a promoter, a ribosome binding site consisting of the nucleotide sequence of SEQ ID NO: 2, a nucleic acid encoding TARS of Akkermansia muciniphila (AmTARS), and a 3′-homology arm.

5. The strain according to claim 4, wherein the promoter is selected from the group consisting of lac promoter, trp promoter, and Tac promoter.

6. The strain according to claim 4, wherein the 5′-homology arm comprises a nucleotide sequence corresponding to a portion of the exo gene and exo/cea intergenic region of Escherichia coli Nissle 1917 (EcN), and the 3′-homology arm comprises a nucleotide sequence corresponding to a portion of the exo/cea intergenic region and cea gene of EcN.

7. The strain according to claim 1, wherein the nucleic acid encoding AmTARS comprises the nucleotide sequence of SEQ ID NO: 1.

8. The strain according to claim 6, wherein the exo gene consists of the nucleotide sequence of SEQ ID NO: 3.

9. The strain according to claim 6, wherein the exo/cea intergenic region consists of the nucleotide sequence of SEQ ID NO: 4.

10. The strain according to claim 6, wherein the cea gene consists of the nucleotide sequence of SEQ ID NO: 5.

11. The strain according to claim 1, wherein the vector further comprises one or more selected from the group consisting of an enhancer, a polyadenylation signal, a Kozak consensus sequence, an ITR (inverted terminal repeat), an LTR (long terminal repeat), a terminator, an internal ribosome entry site (IRES), a fluorescent protein gene, glutathione-S-transferase (GST), horseradish peroxidase (HRP), chloramphenicol acetyltransferase (CAT), beta-galactosidase, beta-glucuronidase, luciferase, histidine (His) tag, V5 tag, FLAG tag, influenza hemagglutinin (HA) tag, Myc tag, 2A self-cleaving peptides, and an antibiotic resistance gene.

12. A pharmaceutical composition for preventing or treating inflammatory diseases comprising the strain according to claim 1 as an active ingredient.

13. The pharmaceutical composition according to claim 12, wherein the inflammatory disease may include inflammatory bowel disease (IBD), edema, dermatitis, conjunctivitis, periodontitis, rhinitis, otitis media, pharyngitis, tonsillitis, pneumonia, gout, ankylosing spondylitis, gastritis, psoriatic arthritis, osteoarthritis, periarthritis of shoulder, tendinitis, tenosynovitis, myositis, hepatitis, lymphangitis, felon, urinary tract infection, peritonitis, cystitis, nephritis, respiratory disease, and sepsis, specifically may be inflammatory bowel disease, and more specifically is one or more selected from the group consisting of colitis, ulcerative colitis, Crohn's disease, and Behcet's enteritis.

14. A food composition for preventing or improving inflammatory diseases comprising the strain according to claim 1 as an active ingredient.

15. A vector comprising a 5′-homology arm, a promoter, a ribosome binding site consisting of the nucleotide sequence of SEQ ID NO: 2, a nucleic acid encoding TARS of Akkermansia muciniphila (AmTARS), and a 3′-homology arm,
wherein the 5′-homology arm comprises a nucleotide sequence corresponding to a portion of the exo gene and exo/cea intergenic region of Escherichia coli Nissle 1917 (EcN),
and the 3′-homology arm comprises a nucleotide sequence corresponding to a portion of the exo/cea intergenic region and cea gene of EcN.

16. A composition for transforming EcN comprising:
the vector according to claim 15;
a guide RNA (gRNA) comprising a sequence complementary to a portion of the exo gene, exo/cea intergenic region, and cea gene sequence of Escherichia coli Nissle 1917 (EcN); and
a nucleic acid encoding a Cas protein.

17. The composition according to claim 16, wherein the Cas protein comprises Cas9, Cas12, Cas13, or variants thereof.

18. A method for producing an EcN strain expressing TARS of Akkermansia muciniphila (AmTARS), comprising treating Escherichia coli Nissle 1917 (EcN) with the composition according to claim 16.

19. The method according to claim 18, wherein the method comprises (a) transforming the Cas protein into EcN; and (b) transforming the gRNA and the vector into EcN.

20. A method for treating inflammatory diseases comprising administering an effective amount of a composition comprising the strain according to claim 1 as an active ingredient to a subject in need thereof.


SEQUENCE LISTING (9 sequences)

SEQ ID NO: 1 - DNA, 1893 bp, synthetic construct
  source 1..1893: mol_type = other DNA
atgtccgaacacaaggaaagaaagactcttgaagaacgccaacagatgtctgatctggaacggctgcgccactcctgcgcgcacgttctggctacggccatttgccgcctctggccggatgcccagctggccggcgggcctgccgtggataacggtttttattacgacgtggagctggaccaccgcatcagcacagaggattttgaacgcattgaggaggagatgaaaaaagtggtgaaggaaaaccaggttttccagaaggaagtcatttcccgcgcggatgccatgaagatggcggaatccggggagctgggcgccctgggcccccgcagcgagccttcccgcttcaagattgacctgctgaacgacatcccggaagacgaggagatttccctctaccgcaacggggattttacggacttgtgcgccggtccccacgtgggccgcacgggcaactgcaaggcattcaaaatcatgagcgtggcgagcgccttctacaagggggacaaaaaccgccccatgctccagcgcatttacggcacctgcttcccgaaccgcacccagcttgacgaacacctggaacggctggaagaggcgcggcgccgcgaccaccgcaaactgggccgcgaactgggcctgttctgcattgacgaatccgtggggcaggggctcatcctctggaagcccaagggggctctcatccgccgttccctgcaggacttcatcacggaagagctggacaagctgggctactcccaggtgtacacgcccaacatcggcaagctggacctgtaccgcacgtccggacacttcccgtattatcaggaaagccagtacgcgcccatcccagaacgggacgccatggaaaaactcagccaggagggggcttcctgcgcggaactgttcaacggcctggccaccggcaccattgagggctatatgctcaagccgatgaactgcccccaccacatcaagatttacgcgaatgacgcccactcctaccgggaccttcccgtgcggctggcggaattcggcacggtgtaccgttgggaacagagcggagagctgggcggcatgacgcgcgtgcgcggtttcacgcaggatgacgcccatatcttctgcacgccggaccagcttgccggggaaatccgccagtgcctgggcatcgtgaaaaccattttcggtacgctgggcatgacggactaccgcgtgcgcctttccatgcgcgacccggaaagcgacaaatacgtgggttctccggaaaattgggacaaagcggaacaggctctgcgggaagctgcggaatggctgggggcggactacagcgaggaagctggggaagccgccttctacggccccaagatcgacttcatcgtgcgcgacgccatcggccgcgaatggcagctgggaaccgtgcaggtggactacaacctgccggaacgctttgacctccattacaccggagcggacaacaagccgcaccggcccgtgatggtgcaccgcgcccccttcggctccatggaacgcttcacggggcttctgattgaacactttgaaggcaaattccccacctggctttccccggaacaggtgcgcgtgcttcccatctccgacaaggtgacggacgtggccgccgcgcatcgaaccgccctggccgccagaggcgtgcgcgtcacggtggacgaaacgccggacaaaataggcgccaaaatccgcaatgcccgtctggaccgcgttccctacatgctggtgctcggccagcgggaggcggaagacggcaccgtctccgtccgccaccgggacaagggggatctgggcgcgatgccctttgagcaattcgcggacctcgtagcccgggaaatcgcggagcgtcatatttctcccgtgatttga

SEQ ID NO: 2 - skipped

SEQ ID NO: 3 - DNA, 448 bp, synthetic construct
  source 1..448: mol_type = other DNA
tgaaaccagactgaagcccacggtcgtcatcgggacgcaggccgccgccgtgcatggtatcagtgagcaggcactctgcggagcaccatcatggactgatgtggtgcggcaactgcgtcatgcaatcggggaccgaccagtaattatctttaatgcccggttcgacatccgcattctgaaaaagactgctgccgcacatagcgatccggctgactggctggaagaactgacggtatattgtgtgatggagctggctgcaggatattatggagcctccaaccgctatggcactatttcactggcctgtgctgccagccagaccggactgaactgggaagggcaggcacactcagcgatcgctgacgcacggatgacggcaggggtggtaaacgctattgctgcatatcatctggaactgctgcaggaacaggcacggctgaaaacctga

SEQ ID NO: 4 - DNA, 76 bp, synthetic construct
  source 1..76: mol_type = other DNA
ctgcctggcctgtataccgcaatcatttacgttatccagacgtaaatatatcaataaatagcaatcccccagatac

SEQ ID NO: 5 - DNA, 240 bp, synthetic construct
  source 1..240: mol_type = other DNA
ctaatgtagttccagcaagcacggccgggcagcttgttctgcctgcgttttcttcaattgagcagtagaccatttagctgtggcatgaatggctgcagaactttcactgttgctacctccagttccaccaccgctgccagagccactcccgtctggattatcattcaaaagagtaatgattacctgccccttatcatcataaggaacaccatctttatagtacgctacagctgtttccat

SEQ ID NO: 6 - DNA, 31 bp, synthetic construct
  source 1..31: mol_type = other DNA
tttacactttatgcttccggctcgtatgttg

SEQ ID NO: 7 - protein, 630 aa, synthetic construct
MSEHKERKTLEERQQMSDLERLRHSCAHVLATAICRLWPDAQLAGGPAVDNGFYYDVELDHRISTEDFERIEEEMKKVVKENQVFQKEVISRADAMKMAESGELGALGPRSEPSRFKIDLLNDIPEDEEISLYRNGDFTDLCAGPHVGRTGNCKAFKIMSVASAFYKGDKNRPMLQRIYGTCFPNRTQLDEHLERLEEARRRDHRKLGRELGLFCIDESVGQGLILWKPKGALIRRSLQDFITEELDKLGYSQVYTPNIGKLDLYRTSGHFPYYQESQYAPIPERDAMEKLSQEGASCAELFNGLATGTIEGYMLKPMNCPHHIKIYANDAHSYRDLPVRLAEFGTVYRWEQSGELGGMTRVRGFTQDDAHIFCTPDQLAGEIRQCLGIVKTIFGTLGMTDYRVRLSMRDPESDKYVGSPENWDKAEQALREAAEWLGADYSEEAGEAAFYGPKIDFIVRDAIGREWQLGTVQVDYNLPERFDLHYTGADNKPHRPVMVHRAPFGSMERFTGLLIEHFEGKFPTWLSPEQVRVLPISDKVTDVAAAHRTALAARGVRVTVDETPDKIGAKIRNARLDRVPYMLVLGQREAEDGTVSVRHRDKGDLGAMPFEQFADLVAREIAERHISPVI

SEQ ID NO: 8 - DNA, 3231 bp, synthetic construct
  source 1..3231: mol_type = other DNA
tgaaaccagactgaagcccacggtcgtcatcgggacgcaggccgccgccgtgcatggtatcagtgagcaggcactctgcggagcaccatcatggactgatgtggtgcggcaactgcgtcatgcaatcggggaccgaccagtaattatctttaatgcccggttcgacatccgcattctgaaaaagactgctgccgcacatagcgatccggctgactggctggaagaactgacggtatattgtgtgatggagctggctgcaggatattatggagcctccaaccgctatggcactatttcactggcctgtgctgccagccagaccggactgaactgggaagggcaggcacactcagcgatcgctgacgcacggatgacggcaggggtggtaaacgctattgctgcatatcatctggaactgctgcaggaacaggcacggctgaaaacctgactgcctggcctgtataccgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagacatcataacagatctggcaaatattctgaaatgagctgttgacaattaatcatcggctcgtataatgtgtggaattgtgagcggataacaatttcacacaggatccacatatgtccgaacacaaggaaagaaagactcttgaagaacgccaacagatgtctgatctggaacggctgcgccactcctgcgcgcacgttctggctacggccatttgccgcctctggccggatgcccagctggccggcgggcctgccgtggataacggtttttattacgacgtggagctggaccaccgcatcagcacagaggattttgaacgcattgaggaggagatgaaaaaagtggtgaaggaaaaccaggttttccagaaggaagtcatttcccgcgcggatgccatgaagatggcggaatccggggagctgggcgccctgggcccccgcagcgagccttcccgcttcaagattgacctgctgaacgacatcccggaagacgaggagatttccctctaccgcaacggggattttacggacttgtgcgccggtccccacgtgggccgcacgggcaactgcaaggcattcaaaatcatgagcgtggcgagcgccttctacaagggggacaaaaaccgccccatgctccagcgcatttacggcacctgcttcccgaaccgcacccagcttgacgaacacctggaacggctggaagaggcgcggcgccgcgaccaccgcaaactgggccgcgaactgggcctgttctgcattgacgaatccgtggggcaggggctcatcctctggaagcccaagggggctctcatccgccgttccctgcaggacttcatcacggaagagctggacaagctgggctactcccaggtgtacacgcccaacatcggcaagctggacctgtaccgcacgtccggacacttcccgtattatcaggaaagccagtacgcgcccatcccagaacgggacgccatggaaaaactcagccaggagggggcttcctgcgcggaactgttcaacggcctggccaccggcaccattgagggctatatgctcaagccgatgaactgcccccaccacatcaagatttacgcgaatgacgcccactcctaccgggaccttcccgtgcggctggcggaattcggcacggtgtaccgttgggaacagagcggagagctgggcggcatgacgcgcgtgcgcggtttcacgcaggatgacgcccatatcttctgcacgccggaccagcttgccggggaaatccgccagtgcctgggcatcgtgaaaaccattttcggtacgctgggcatgacggactaccgcgtgcgcctttccatgcgcgacccggaaagcgacaaatacgtgggttctccggaaaattgggacaaagcggaacaggctctgcgggaagctgcggaatggctgggggcggactacagcgaggaagctggggaagccgccttctacggccccaagatcgacttcatcgtgcgcgacgccatcggccgcgaatggcagctgggaaccgtgcaggtggactacaacctgccggaacgctttgacctccattacaccggagcggacaacaagccgcaccggcccgtgatggtgcaccgcgcccccttcggctccatggaacgcttcacggggcttctgattgaacactttgaaggcaaattccccacctggctttccccggaacaggtgcgcgtgcttcccatctccgacaaggtgacggacgtggccgccgcgcatcgaaccgccctggccgccagaggcgtgcgcgtcacggtggacgaaacgccggacaaaataggcgccaaaatccgcaatgcccgtctggaccgcgttccctacatgctggtgctcggccagcgggaggcggaagacggcaccgtctccgtccgccaccgggacaagggggatctgggcgcgatgccctttgagcaattcgcggacctcgtagcccgggaaatcgcggagcgtcatatttctcccgtgatttgagatccggtcgactcgagcggccgcatcgtgactgactgacgatctgcctcgcgcgtttcggtgatgacggtgaaaacctctgacacatgcagctcccggagacggtcacagcttgtctgtaagcggatgccatttacgttatccagacgtaaatatatcaataaatagcaatcccccagatacctaatgtagttccagcaagcacggccgggcagcttgttctgcctgcgttttcttcaattgagcagtagaccatttagctgtggcatgaatggctgcagaactttcactgttgctacctccagttccaccaccgctgccagagccactcccgtctggattatcattcaaaagagtaatgattacctgccccttatcatcataaggaacaccatctttatagtacgctacagctgtttccattataaaatcctctttgacattaaaaacaatcagttaaaaataagtactgcatatataattactggttttatatacagcataaaaattacgccgctgcgttttccctgtcaaccctg

SEQ ID NO: 9 - DNA, 20 bp, synthetic construct
  source 1..20: mol_type = other DNA
caatcatttacgttatccag